(19) United States
(12) Patent Application Publication
(10) Pub. No.: US 2016/0317600 A1
(43) Pub. Date: Nov. 3, 2016

(54) NUTRITIONAL COMPOSITION COMPRISING HYDROLYZED PROTEIN

(71) Applicant: ABBOTT LABORATORIES, Abbott Park, IL (US)

(72) Inventors: PAUL JOHNS, Columbus, OH (US); CYNTHIA NEAL, Pickerington, OH (US); JOHN MCEWEN, Columbus, OH (US); RONALD MCKENNA, Lewis Center, OH (US)

(73) Assignee: ABBOTT LABORATORIES, Abbott Park, IL (US)

(21) Appl. No.: 15/105,059

(22) PCT Filed: Dec. 18, 2014
(86) PCT No.: PCT/US2014/071236

§ 371 Date: Jun. 16, 2016

Related U.S. Application Data
(60) Provisional application No. 61/918,121, filed on Dec. 19, 2013.

(51) Int. Cl. A61K 38/01; A61K 33/34; A61K 31/202; A61K 9/00; A23L 33/12; A23L 33/115; A23L 33/00; A23L 33/15; A23L 33/125; A23L 33/18; A23L 33/21; A61K 31/375; A23L 33/19

(52) U.S. Cl. CPC A61K 38/018; A61K 31/375; A61K 33/34; A61K 31/202; A61K 9/0095; A23L 33/12; A23L 33/19; A23L 33/30; A23L 33/15; A23L 33/125; A23L 33/18; A23L 33/21; A23L 33/115; A23L 33/40; A23V 2002/00

(57) ABSTRACT

Liquid nutritional compositions include specific combinations of protein, carbohydrate, and fat for the nutritional support of individuals with particular needs, such as individual suffering from obesity. The compositions comprise a source of at least one omega-6 fatty acid and a source of at least one omega-3 fatty acid with a ratio of omega-6 to omega-3 of 1:5 to 1:1. In a preferred form the compositions comprise at least one hydrolysed dairy protein selected from hydrolysed whey protein and hydrolysed casein protein or mixtures thereof. Methods of administering such nutritional compositions are also provided. Further liquid nutritional compositions comprise protein in an amount of 1-12% by weight of the liquid nutritional composition, vitamin C and copper, wherein the protein comprises a mixture of whey protein and casein with a majority of the whey protein having a degree of hydrolysis greater than 20%. Methods for improving vitamin C stability in these liquid nutritional compositions are also provided.

### CROSS-REFERENCE TO RELATED APPLICATIONS

This application claims priority to and any benefit of U.S. Provisional Application No. 61/918,121, filed Dec. 19, 2013, the content of which is incorporated herein by reference in its entirety.

### TECHNICAL FIELD

The general inventive concepts relate to liquid nutritional compositions and more particularly to liquid nutritional compositions with a novel macronutrient profile.

### BACKGROUND

Use of nutritional compositions, and more particularly nutritional liquids, for providing various individuals with sole or supplemental nutrition is known. These compositions most often contain varying amounts and types of proteins, carbohydrate, lipids, vitamins, and minerals, all depending largely upon the nutritional needs of the intended user.

Many such nutritional compositions are used by consumers to provide support for a healthy lifestyle or for anabolic results (i.e., increasing muscle mass). Certain other consumers use the nutritional compositions for maintenance of lean muscle mass. This is especially true for consumers that are obese, have impaired digestive function, or that are critically ill. These consumers require specially tailored nutrition with particular macronutrient delivery in order to maintain lean muscle mass, and in the case of critically ill obese subjects, limiting the amount of calories in the nutritional composition from fat.

In addition, certain consumers with impaired digestive systems, or other condition that might affect protein absorption, are able to absorb particular forms of protein better than others. For some of these consumers, hydrolyzed proteins are better suited to meet their nutritional needs. However, certain hydrolyzed proteins can increase the natural degradation of important nutrients, especially in liquid nutritional compositions, often leading to reduced shelf stability. Vitamin C is one such nutrient. Certain liquid nutritional compositions that include both hydrolyzed protein and vitamin C demonstrate reduced amounts of vitamin C relative to initial fortification. In concert with this reduced vitamin C, liquid nutritional compositions that include both hydrolyzed protein and vitamin C also demonstrate increased development of unwanted discoloration (also called Maillard browning).

### SUMMARY

Provided herein are nutritional compositions with novel macronutrient profiles. The liquid nutritional compositions can be used for the preservation of lean muscle mass in an individual. In certain exemplary embodiments, these compositions are tailored to meet the needs of individuals seeking to maintain lean muscle mass. In certain exemplary embodiments, the nutritional compositions are tailored to meet the nutritional needs of critically ill obese subjects.

In a first exemplary embodiment, a liquid nutritional composition comprising protein, carbohydrate, and fat is provided. The fat is present in an amount of 15 to 25% of the total calories of the liquid nutritional composition. The fat comprises a source of at least one omega-6 fatty acid and a source of at least one omega-3 fatty acid, with a ratio of omega-6 to omega-3 fatty acids of 1:5 to 1:1.

In a second exemplary embodiment, a liquid nutritional composition is provided. The liquid nutritional composition comprises: protein present in an amount of 35% of the total calories of the liquid nutritional composition, the protein comprising a source of at least one dairy protein selected from extensively hydrolyzed whey protein and slightly hydrolyzed casein protein; carbohydrate present in an amount of 45% of the total calories of the liquid nutritional composition, the carbohydrate comprising less than 1 g/L of indigestible carbohydrate; and fat present in an amount of 20% of the total calories of the liquid nutritional composition, the fat comprising a source of at least one omega-3 fatty acid selected from eicosapentaenoic acid, docosahexaenoic acid, and combinations thereof, in an amount of 2 g/L to 5 g/L of the liquid nutritional composition, and the fat comprising a source of at least one omega-6 fatty acid. The fat has a ratio of omega-6 to omega-3 fatty acids of 1:4.

In a third exemplary embodiment, a method of treating a subject suffering from obesity is provided. The method comprises administering a liquid nutritional composition to a subject suffering from obesity, the liquid nutritional composition comprising protein, carbohydrate, and fat, the fat present in an amount of 15 to 25% of the total calories of the liquid nutritional composition. The fat comprises a source of at least one omega-6 fatty acid and a source of at least one omega-3 fatty acid, with a ratio of omega-6 to omega-3 fatty acids of 1:5 to 1:1.

In a fourth exemplary embodiment, a composition useful for treating a subject suffering from obesity is provided. The composition comprises protein present in an amount of 30 to 40% of the total calories of the liquid nutritional composition, and comprising a source of at least one dairy protein selected from hydrolyzed whey protein and hydrolyzed casein protein; carbohydrate present in an amount of 35 to 55% of the total calories of the liquid nutritional composition, and comprising less than 1 g/L of indigestible carbohydrate; and fat present in an amount of 15 to 25% of the total calories of the composition. The fat comprises a source of at least one omega-6 fatty acid and a source of at least one omega-3 fatty acid, with a ratio of omega-6 to omega-3 fatty acids of 1:5 to 1:1.

In a fifth exemplary embodiment, a liquid nutritional composition is provided. The liquid nutritional composition comprises protein in an amount of 1 to 12% by weight of the liquid nutritional composition, vitamin C in an amount of 0.01 to 0.2% by weight of the liquid nutritional composition, and copper in an amount of 0.00005 to 0.001% by weight of the liquid nutritional composition, wherein the copper is copper sulfate pentahydrate, wherein the protein comprises 60 to 80% whey protein and 20 to 40% casein by weight of the protein in the liquid nutritional composition, with a majority of the whey protein having a degree of hydrolysis greater than 20%.

In a sixth exemplary embodiment, a method for improving vitamin C stability in a liquid nutritional composition is provided. The method comprises providing hydrolyzed whey protein in an amount of 1 to 10% by weight of the liquid nutritional composition, a majority of the hydrolyzed whey protein having a degree of hydrolysis greater than 20%; providing a combination of vitamin C in an amount of 0.01 to 0.02% by weight of the liquid nutritional composition and copper in an amount of 0.00005 to 0.001% by weight of the liquid nutritional composition, wherein the copper is provided in a form that restricts its availability for oxidative processes; and mixing the hydrolyzed whey protein, the vitamin C, and the copper to form a liquid nutritional composition.

In a seventh exemplary embodiment, a method for improving vitamin C stability in a liquid nutritional composition is provided. The method comprises providing hydrolyzed whey protein in an amount of 1 to 10% by weight of the liquid nutritional composition, a majority of the hydrolyzed whey protein having a degree of hydrolysis greater than 20%; providing a combination of a salt of vitamin C in an amount of 0.01 to 0.2% by weight of the liquid nutritional composition, providing copper sulfate pentahydrate in an amount of 0.00005 to 0.001% by weight of the liquid nutritional composition; and mixing the hydrolyzed whey protein, the salt of vitamin C and the copper sulfate pentahydrate to form a liquid nutritional composition.

In an eighth exemplary embodiment, a method for improving vitamin C stability in a liquid nutritional composition is provided. The method comprises providing hydrolyzed whey protein in an amount of 1 to 10% by weight of the liquid nutritional composition, a majority of the hydrolyzed whey protein having a degree of hydrolysis greater than 20%; providing a combination of vitamin C in an amount of 0.01 to 0.2% by weight of the liquid nutritional composition and copper in an amount of 0.00005 to 0.001% by weight of the liquid nutritional composition; and mixing the hydrolyzed whey protein, the vitamin C, and the copper to form a liquid nutritional composition, wherein the stability of the vitamin C is enhanced in the liquid nutritional composition.

In a ninth exemplary embodiment, a method for improving vitamin C stability in a liquid nutritional composition is provided. The method comprises providing hydrolyzed whey protein in an amount of 1 to 10% by weight of the liquid nutritional composition, a majority of the hydrolyzed whey protein having a degree of hydrolysis greater than 20%; providing vitamin C in an amount of 0.01 to 0.2% by weight of the liquid nutritional composition; providing copper in an amount of 0.00005 to 0.001% by weight of the liquid nutritional composition; mixing the hydrolyzed whey protein, the vitamin C, and the copper to form a liquid nutritional composition; reducing the level of available oxygen above the liquid nutritional composition during mixing by providing a substantially unreactive gas in a headspace of the liquid nutritional composition during mixing.

### BRIEF DESCRIPTION OF THE DRAWINGS

FIG. 1 shows a graph of the percent recovery of vitamin C from exemplary compositions including hydrolyzed protein.

### DETAILED DESCRIPTION

Provided herein are nutritional compositions with novel macronutrient profiles. Certain exemplary embodiments provide nutritional compositions for maintaining lean muscle mass (including preventing muscle loss) in an individual. Certain exemplary embodiments provide this benefit through particular protein, carbohydrate, and fat compositions or ratios. Certain exemplary embodiments provide therapeutic nutritional compositions including high protein and low fat, and particularly formulated for individuals with malabsorption, maldigestion, impaired gastrointestinal (GI) function, the critically ill obese individual, or individuals with combinations of these conditions. Certain exemplary embodiments provide compositions useful for treating an individual suffering from obesity, as well as methods for treating an individual suffering from obesity.

In addition, certain consumers with impaired digestive systems, or other conditions that might affect protein absorption, are often able to absorb particular forms of protein better than others. For some of these consumers, hydrolyzed proteins are better suited to meet their nutritional needs. However, certain hydrolyzed proteins (e.g., hydrolyzed whey protein) can increase the degradation of important nutrients, especially in liquid nutritional compositions. Vitamin C is one such nutrient. Conventional liquid nutritional compositions that included both hydrolyzed proteins and vitamin C also demonstrate increased development of unwanted discoloration, often brown in color. While not wishing to be bound by theory, it is believed that interactions between hydrolyzed protein, vitamin C, and copper in liquid nutritional compositions contribute both to the degradation of vitamin C and development of unwanted discoloration. Provide herein are, liquid nutritional compositions with improved vitamin C stability as well as methods for improving the stability of C in a liquid nutritional composition.

The term “nutritional composition” as used herein, unless otherwise specified, refers to nutritional products in various forms including, but not limited to, liquids, solids, powders, semi-liquids, semi-solids, nutritional supplements, and any other nutritional food product known in the art. A nutritional composition in powder form may often be reconstituted to form a nutritional composition in liquid form. The nutritional compositions disclosed herein are generally suitable for oral consumption by a human.

The terms “nutritional powder” and “reconstitutable powder” as used herein, unless otherwise specified, refer to nutritional compositions in flowable or scoopable form that can be reconstituted with water or another aqueous liquid prior to consumption and include both spray dried and drymixed/dryblended powders.

The term “nutritional liquid” as used herein, unless otherwise specified, refers to nutritional compositions in ready-to-drink liquid form, concentrated liquid form, and nutritional liquids made by reconstituting nutritional powders described herein prior to use. The nutritional liquid may also be formulated as a suspension, an emulsion, a solution, and so forth.

The terms “administer,” “administering,” “administered,” and “administration” as used herein, unless otherwise specified, should be understood to include providing a composition to an individual, the act of consuming a composition by an individual, and combinations thereof.

The term “serving” as used herein, unless otherwise specified, refers to any amount which is intended to be consumed by an individual in one sitting or within one hour or less.

The compositions according to certain exemplary embodiments include those suitable for oral administration. Oral administration, as defined herein, includes any form of administration in which the composition passes through the esophagus of the individual. For example, oral administration includes nasogastric intubation, in which a tube is run from through the nose to the stomach of the individual to administer food or drugs.

The term “muscle” as used herein, unless otherwise specified, refers to skeletal muscle and other non-skeletal, striated muscles such as diaphragm, extraocular muscle, and so forth.

The term “muscle mass” as used herein, unless otherwise specified, refers to the amount or size of muscle or muscle groups, as expressed by muscle weight, mass, area, or volume. Muscle mass may also be expressed as total lean body mass, lean body mass of a body compartment such as the leg, or cross-sectional area of a leg or arm compartment.

The term “muscle loss” as used herein, unless otherwise specified, refers to a loss of muscle mass (also known as muscle wasting). Muscle loss may be caused by normal aging (e.g., sarcopenia), inactivity (e.g., muscle disuse or immobility), or chronic disease or disease-related disorders (e.g., wasting, cancer, cachexia).

The terms “treatment,” “treat,” and “to alleviate” as used herein, unless otherwise specified, include both prophylactic or preventive treatment (that prevent or slow the development of a targeted pathologic condition or disorder) and curative, therapeutic or disease-modifying treatment, including therapeutic measures that cure, slow down, lessen symptoms of, or halt progression of a diagnosed pathologic condition or disorder; and treatment of individuals at risk of contracting a disease or suspected to have contracted a disease, as well as individuals who are ill or have been diagnosed as suffering from a disease or medical condition. The term does not necessarily imply that an individual is treated until total recovery. The terms “treatment” and “treat” also refer to the maintenance or promotion of health in an individual not suffering from a disease but who may be susceptible to the development of an unhealthy condition, such as nitrogen imbalance or muscle loss. The terms “treatment,” “treat,” and “to alleviate” are also intended to include the potentiation or enhancement of one or more primary prophylactic or therapeutic measures. The terms “treatment,” “treat,” and “to alleviate” are further intended to include the dietary management of a disease or condition or the dietary management for prophylaxis or prevention of a disease or condition.

The term “critically ill obese” as used herein, unless otherwise specified, refers to an individual with a Body Mass Index (BMI) of greater than or equal to 30 kg/m², and in certain instances refers to a patient with a critical illness or condition requiring, for example extended bed rest, ventilation, tube feeding, multiple surgical procedures, extended hospital stays, and the like.

The term “absorbed” or “absorption” as used herein, unless otherwise specified, when referring to protein, carbohydrate, or fat refers to the rate that the macronutrient is digested after consumption by an individual.

In certain exemplary embodiments, the nutritional composition is a nutritional liquid. Non-limiting examples of nutritional liquids include snack and meal replacement products; hot or cold beverages; carbonated or non-carbonated beverages; juices or other acidified beverages; milk or soy-based beverages; shakes; coffees; teas; compositions for administration by nasogastric intubation; and so forth. Generally, the nutritional liquids are formulated as suspensions or emulsions, but the nutritional liquids can also be formulated in any other suitable form such as clear liquids, solutions, liquid gels, liquid yogurts, frozen pops, and so forth.

In certain exemplary embodiments, the nutritional composition is manufactured as a reconstitutable nutritional powder. The nutritional powders may be reconstituted with water prior to use to a caloric density tailored to the nutritional needs of the ultimate user, although in most instances the compositions comprise from 100 calories per serving to 500 calories per serving, including from 150 calories per serving to 350 calories per serving, and also including from 200 calories per serving to 320 calories per serving.

Generally speaking, lean body mass, and in particular muscle mass, is in a constant state of turnover, where the competing processes of muscle protein synthesis (MPS) and muscle protein degradation (MPD) take place. Changes in lean body mass can be viewed as an equation, such as:

Change in lean body mass=(rate of MPS)−(rate of MPD)

If the value is positive (i.e., if MPS is greater than MPD), then lean body mass increases. Conversely, if the value is negative then lean body mass decreases. If MPS is equal to MPD lean body mass can be maintained. Therefore, in order to increase or maintain lean muscle mass, MPS must be greater than or equal to MPD.

Maintaining a healthy ratio of lean muscle mass to fat mass is important in achieving a healthy lifestyle. This is also true for individuals with malabsorption, maldigestion, impaired gastrointestinal (GI) function, and obese (including critically ill obese) individuals. Increased amounts of fat in nutritional compositions can decrease gastric emptying time and can lead to increases in adipose deposition, especially in obese patients. This increased adipose deposition leads to a variety of unhealthy outcomes for the individual. In addition, inadequate dietary intake of protein can cause undesirable health complications as well.

As such, it is beneficial to provide a sufficient amount of protein to an individual for maintenance of lean muscle mass, while avoiding any overfeeding complications that can be associated with administration of nutritional compositions with increased amounts of fat.

One common therapy for individuals suffering from malabsorption, maldigestion, impaired gastrointestinal (GI) function, and obesity (including critically ill obese individuals), includes the administration of one or more nutritional compositions to help meet the nutritional needs of the particular individual. However, conventional nutritional compositions often lack one or more particularly useful nutrients or include nutrient profiles that do not properly meet the needs of the particular individual. Provided herein are improved nutritional compositions that better meet the needs of individuals suffering from conditions such as malabsorption, maldigestion, impaired gastrointestinal (GI) function, and obesity. Certain exemplary embodiments provide compositions useful for the treatment of critically ill obese individuals.

Certain exemplary embodiments encompass nutritional compositions having or providing of one or more of the following properties:

Low fat content in combination with high protein content;

Improved GI tolerance through the inclusion of a particular blend of medium chain triglycerides and long chain triglycerides;

About 50% by weight of fat as medium chain triglycerides;

About 15 to 25% of total calories from fat;

Eicosapentaenoic acid (EPA) and docosahexaenoic acid (DHA) as a source of fat;

EPA in an amount of about 2.5 g/L of the nutritional composition;

DHA in an amount of about 1.07 g/L of the nutritional composition;

Fish oil in an amount of about 9.2 g/L of the nutritional composition;

A blend of polyunsaturated and monounsaturated fatty acids;

A ratio of omega-6 to omega-3 fatty acids of 1:5 to 1:1;

A good source of plant-based unsaturated fatty acids, fish based unsaturated fatty acids, or combinations thereof, to support heart and circulatory health;

Nutritional support for the acutely obese patient according to the A.S.P.E.N. Clinical Guidelines: Nutritional Support of Hospitalized Adult Patients with Obesity;

Improved GI tolerance through the inclusion of hydrolyzed protein;

About 30 to 40% of total calories from protein;

A protein blend that includes a source of essential amino acids to promote nitrogen utilization and retention, to minimize the need for modular protein supplements, and to support healing;

At least one of casein and whey proteins;

About 35 to 55% of total calories from carbohydrate;

A caloric density of about 1000 kcal/L;

Elevated levels of antioxidants such as vitamin C and vitamin E;

Little to no fiber;

Lactose in an amount of less than 500 mg/L of the nutritional composition;

Vitamin D in an amount of about 240 IU per 8 oz., and in certain embodiments, from about 1100 to about 1600 IU per liter of the nutritional composition;

Nutritional support for individuals who suffer from at least one of acute obesity, mechanical ventilation, pancreatitis, malnutrition, malabsorption, critical illness with GI impairment.

It is intended that when discussing the liquid nutritional compositions disclosed herein, that the discussion may apply equally to the methods disclosed herein, especially in those instances where the methods relate to administration of such liquid nutritional compositions.

In certain exemplary embodiments, the nutritional composition comprises protein, carbohydrate, and fat. The protein, carbohydrate, and fat contribute to the total caloric density of the liquid nutritional composition. In certain exemplary embodiments, the liquid nutritional composition has a caloric density of about 1 kcal per mL.

In certain exemplary embodiments, the liquid nutritional composition comprises protein or at least one source of protein. The particular amount of protein present in the nutritional composition may vary depending upon the nutritional needs of the intended user. In certain exemplary embodiments, protein is present in an amount of 15 to 40% of the total calories in the liquid nutritional composition. In certain exemplary embodiments, protein is present in an amount of 30 to 40% of the total calories of the liquid nutritional composition. In certain exemplary embodiments, protein is present in an amount of not more than about 35% of the total calories of the liquid nutritional composition. In certain exemplary embodiments, the protein of the nutritional composition comprises at least one dairy protein.

The dairy protein may be provided by a single source or a combination of sources. In certain exemplary embodiments, the liquid nutritional composition comprises a source of dairy protein comprising both whey protein and casein. In certain exemplary embodiments, the liquid nutritional composition comprises at least one of a hydrolyzed whey protein and a hydrolyzed casein protein. In this context, the terms “protein hydrolysates” or “hydrolyzed protein” are used interchangeably herein and include slightly hydrolyzed protein and extensively hydrolyzed proteins. Slightly hydrolyzed proteins include those proteins wherein the DH is most often 0.1% to 10%. Extensively hydrolyzed proteins include those proteins wherein the degree of hydrolysis is most often at least about 20%, including from about 20% to about 70%. In certain exemplary embodiments, extensively hydrolyzed proteins include those proteins wherein the degree of hydrolysis is from about 30% to about 60%. In certain exemplary embodiments, extensively hydrolyzed proteins include those proteins wherein the degree of hydrolysis is about 50 to 60%. The degree of protein hydrolysis for purposes of characterizing the hydrolyzed protein component of the liquid nutritional compositions is easily determined by one of ordinary skill in the formulation arts, for example, by quantifying the amino nitrogen to total nitrogen ratio (AN/TN) of the protein component of the selected liquid formulation. The amino nitrogen component is quantified by USP titration methods for determining amino nitrogen content, while the total nitrogen component is determined by the Tecator Kjeldahl method, all of which are well known methods to one of ordinary skill in the analytical chemistry art.

In certain exemplary embodiments, the liquid nutritional composition comprises extensively hydrolyzed whey protein. The extensively hydrolyzed whey protein may have a DH of from about 20 to 70%. Commercial sources of hydrolyzed whey protein are well known in the nutrition art, non-limiting examples of which include a hydrolyzed whey protein available from Fonterra Co-operative Group.

In certain exemplary embodiments, the liquid nutritional composition comprises slightly hydrolyzed casein protein. In certain exemplary embodiments, the slightly hydrolyzed casein is sodium caseinate. The slightly hydrolyzed casein protein may have a DH of from 0.1 to 10%. Commercial sources of hydrolyzed casein protein are well known in the nutrition art, non-limiting examples of which include a slightly hydrolyzed casein protein available from Fonterra Co-operative Group. In certain exemplary embodiments, the liquid nutritional composition comprises extensively hydrolyzed whey protein and a slightly hydrolyzed casein. In certain exemplary embodiments, the liquid nutritional composition comprises extensively hydrolyzed protein and a slightly hydrolyzed casein in a ratio of 1:1 to 5:1. In certain exemplary embodiments, the liquid nutritional composition comprises extensively hydrolyzed protein and a slightly hydrolyzed casein in a ratio of about 2:1.

In addition to the protein sources discussed above, in certain exemplary embodiments, the nutritional composition may also contain protein from one or more other sources, in an amount or amounts that is in accordance with the guidance on the amounts of proteins noted above. The one or more other sources of protein may in certain embodiments include animal products (e.g., other dairy proteins, meat, fish, egg albumen), cereals (e.g., rice, corn), vegetables (e.g., soy, pea, potato), and combinations thereof. The additional protein sources can also include, free amino acids known for use in nutritional compositions, non-limiting examples of which include L-tryptophan, L-glutamine, L-tyrosine, L-methionine, L-cysteine, L-arginine, and combinations thereof.

In certain exemplary embodiments, the liquid nutritional composition comprises carbohydrate or at least one source of carbohydrate. The particular amount of carbohydrate present in the nutritional composition may vary depending upon the nutritional needs of the intended user. In certain exemplary embodiments, the liquid nutritional composition comprises carbohydrate in an amount of 35 to 55% of the calories in the liquid nutritional composition. In certain exemplary embodiments, the liquid nutritional composition comprises carbohydrate in an amount of at least about 40% of the calories in the liquid nutritional composition. In certain exemplary embodiments, the liquid nutritional composition comprises carbohydrate in an amount of about 45% of the calories in the liquid nutritional composition.

Sources of carbohydrate which may be suitable for use in certain exemplary embodiments of the nutritional compositions disclosed herein include simple, complex, or variations and combinations thereof. Generally, any source of carbohydrate may be used so long as it is suitable for use in oral nutritional compositions and is otherwise compatible with any other selected ingredients or features present in the nutritional composition. Non-limiting examples of carbohydrate or sources thereof which may be suitable for use in the nutritional compositions disclosed herein include maltodextrin; hydrolyzed or modified starch or cornstarch; glucose polymers; corn syrup; corn syrup solids; rice-derived carbohydrates; sucrose; glucose; fructose; lactose; high fructose corn syrup; honey; and sugar alcohols (e.g., maltitol, erythritol, sorbitol). In certain exemplary embodiments, the carbohydrate has a DE of 2 to 20.

In certain exemplary embodiments, the carbohydrate or carbohydrate component of the nutritional composition comprises slowly digested carbohydrate or starches in addition to the more readily digestible carbohydrates discussed above. Exemplary sources of such carbohydrates include: isomaltulose; sucromalt; pullulan; potato starch and other slowly-digested carbohydrates, dietary fibers including, but not limited to, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low and high methoxy pectin, cereal beta-glucans (i.e., oat beta-glucan, barley beta-glucan), carrageenan, psyllium, Fibersol™, and other resistant starches; and combinations thereof. In certain exemplary embodiments, the liquid nutritional composition comprises 0 g/L to 1 g/L of indigestible carbohydrate. In certain exemplary embodiments, the liquid nutritional composition is substantially free of fiber.

In certain exemplary embodiments, the nutritional composition comprises fat, or at least one source of fat. The particular amount of fat present in the nutritional composition may vary depending upon the nutritional needs of the intended user. In certain exemplary embodiments, fat is present in an amount of 15 to 25% of the total calories of the liquid nutritional composition. In certain exemplary embodiments, fat is present in an amount of not more than about 20% of the total calories of the liquid nutritional composition. In certain exemplary embodiments, fat is present in an amount of about 20% of the total calories of the liquid nutritional composition. In certain exemplary embodiments, the fat comprises a ratio of medium chain triglyceride (MCT) to long chain triglyceride (LCT) of about 1:1. Non-limiting examples of fats or sources thereof which may be suitable for use in the nutritional compositions include coconut oil, fractionated coconut oil, soy oil, corn oil, olive oil, safflower oil, high oleic safflower oil, MCT (medium chain triglycerides) oil, sunflower oil, high oleic sunflower oil, palm and palm kernel oils, palm olein, canola oil, marine oils, cottonseed oils, non-dairy creamer, and combinations thereof.

In certain exemplary embodiments, the nutritional composition comprises unsaturated fat or a source of unsaturated fat. In certain exemplary embodiments, the source of unsaturated fat comprises fish oil. The fish oil may be a source of monounsaturated fat, a source of polyunsaturated fat, or both. In certain exemplary embodiments, the fat comprises a source of at least one omega-6 fatty acid. In certain exemplary embodiments, the fat comprises a source of at least one omega-3 fatty acid. In certain exemplary embodiments, the nutritional composition comprises a ratio of omega-6 to omega-3 fatty acids of 1:5 to 1:1. In certain exemplary embodiments, the nutritional composition comprises a ratio of omega-6 to omega-3 fatty acids of 1:4.5 to 1:3. In certain exemplary embodiments, the nutritional composition comprises a ratio of omega-6 to omega-3 fatty acids of about 1:4. In certain exemplary embodiments, the fat in the nutritional composition comprises a source of at least one of EPA and DHA. In certain exemplary embodiments, the fat in the nutritional composition comprises alpha linolenic acid (ALA) or a source of ALA. The amount of EPA and DHA present in the liquid nutritional compositions of the exemplary embodiments may also be expressed as a total amount of the two fatty acids per liter of the liquid nutritional composition. In certain exemplary embodiments, the liquid nutritional composition comprises at least one of EPA, DHA, and combinations thereof, in an amount of at least about 2 g/L of the liquid nutritional composition. In certain exemplary embodiments, the liquid nutritional composition comprises at least one of EPA, DHA, and combinations thereof, in an amount of 2 g/L to 5 g/L of the liquid nutritional composition. In certain exemplary embodiments, the liquid nutritional composition comprises at least one of EPA, DHA, and combinations thereof, in an amount of about 3.5 g/L of the liquid nutritional composition. In certain exemplary embodiments, the liquid nutritional composition comprises EPA in an amount of about 2.5 g/L of the nutritional composition. In certain exemplary embodiments, the liquid nutritional composition comprises DHA in an amount of about 1.07 g/L of the nutritional composition. In certain exemplary embodiments, the liquid nutritional composition comprises EPA in an amount of about 2.5 g/L of the nutritional composition, and DHA in an amount of about 1.07 g/L of the nutritional composition.

In certain exemplary embodiments, the liquid nutritional compositions comprise at least one antioxidant. Antioxidants suitable for use in the liquid nutritional composition include carotenoids, coenzyme Q10 (“CoQIO”), flavonoids, glutathione Goji (wolfberry), hesperidine, lactowolfberry, lignan, lutein, lycopene, polyphenols, selenium, vitamin A, vitamin B₁, vitamin B₆, vitamin B₁₂, vitamin C, vitamin D, vitamin E, and combinations thereof. In certain exemplary embodiments, the liquid nutritional composition comprises vitamin C in an amount of about 500 to about 800 mg per liter of the nutritional composition. In certain exemplary embodiments, the liquid nutritional composition comprises vitamin E in an amount of about 250 IU to about 310 IU per liter of the nutritional composition.

Vitamin D forms include Vitamin D2 (ergocalciferol), Vitamin D3 (cholecalciferol), and any other forms suitable for use in a nutritional product. The amount of Vitamin D in the nutritional composition typically ranges up to about 1200 IU to about 1600 IU per liter of the nutritional composition. In certain exemplary embodiments, the nutritional composition comprises Vitamin D in an amount of about 240 IU per 8 oz. of the nutritional composition.

Individuals suffering from conditions such as acute obesity, mechanical ventilation, pancreatitis, malnutrition, malabsorption, critical illness with GI impairment; maldigestion, and impaired gastrointestinal (GI) function, and critically ill obese individuals, require particular nutritional support. Accordingly, provided herein are methods for treating a subject suffering from one or more of the above conditions. In certain exemplary embodiments, a method of treating a subject suffering from obesity comprises administering a liquid nutritional composition to the subject suffering from obesity, the liquid nutritional composition comprising protein, carbohydrate, and fat, wherein the fat is present in an amount of 15 to 25% of the total calories of the liquid nutritional composition, with a ratio of omega-6 to omega-3 fatty acids of 1:5 to 1:1.

Related to these methods are compositions useful for treating an individual suffering from obesity. In certain exemplary embodiments, a liquid nutritional composition useful for treating a subject suffering from obesity comprises protein present in an amount of 30 to 40% of the total calories of the liquid nutritional composition, the protein comprising a source of at least one dairy protein selected from hydrolyzed whey protein and hydrolyzed casein protein; carbohydrate present in an amount of 35 to 55% of the total calories of the liquid nutritional composition, the carbohydrate comprising less than 1 g/L of indigestible carbohydrate; and fat present in an amount of 15 to 25% of the total calories of the liquid nutritional composition, and a ratio of omega-6 to omega-3 fatty acids of 1:5 to 1:1.

As noted above, liquid nutritional compositions that comprise hydrolyzed whey protein are known to suffer from degradation of one or more vitamins. This degradation often presents as a brown discoloration in the composition. Vitamin C is one vitamin that is known to demonstrate poor stability in liquid nutritional compositions. It has been observed that liquid nutritional compositions that demonstrate poor vitamin stability and development of discoloration, generally include three components: 1) hydrolyzed protein (including hydrolyzed whey protein), 2) vitamin C, and 3) copper. While not wishing to be bound by theory, it is believed that interactions between the hydrolyzed protein, the vitamin C, and the copper lead to both vitamin C degradation and unwanted browning. Therefore, provided herein are compositions and methods that minimize the interactions between these three elements in order to reduce these phenomenon. Generally, the methods seek to restrict the participation of copper, ascorbic acid, and oxygen that are implicated in the vitamin C decomposition and the associated unwanted discoloration. The general inventive concepts encompass liquid nutritional compositions that comprise hydrolyzed whey protein and vitamin C.

Table 1 shows the relative vitamin C recovery in the presence of hydrolyzed casein and whey proteins, both with and without the addition of copper. The samples were prepared in aqueous solutions in 0.10 M HEPES, pH 6.8, in restricted headspace glass vials, with the protein component present in a concentration of 22.6 g/L, with the vitamin C present in a concentration of 297 mg/L. Vitamin C recovery was measured by HPLC after 16 hrs at room temperature.

TABLE 1

| Protein | DH of | Amount of Copper | Vitamin C as % of |
|---|---|---|---|
| Hydrolysate | Protein | (added as CuSO₄•5H₂0) | Total Added |
| Casein | 38 | 0 | 58.4 |
| | | 2 mg/L | 40.5 |
| Whey | 27 | 0 | 70.9 |
| | | 2 mg/L | 38.8 |

Table 2 shows the relative vitamin C recovery in the presence of whey protein with varied degrees of hydrolysis, both with and without the presence of copper. The samples were prepared in aqueous solutions in 0.10 M HEPES, pH 6.8, in restricted headspace glass vials, with the protein component present in a concentration of 22.6 g/L, with the vitamin C present in a concentration of 297 mg/L. Vitamin C recovery was measured by HPLC after 16 hrs at room temperature.

TABLE 2

| Protein | DH of | Amount of Copper | Vitamin C as % of |
|---|---|---|---|
| Hydrolysate | protein | (added as CuSO₄•5H₂0) | total added |
| WP1 | 0 | 0 | 86.5 |
| | | 2 mg/L | 61.4 |
| WP2 | 7 | 0 | 86 |
| | | 2 mg/L | 54.6 |
| WP3 | 15 | 0 | 79.9 |
| | | 2 mg/L | 45.8 |
| WP4 | 27 | 0 | 74.4 |
| | | 2 mg/L | 43.9 |

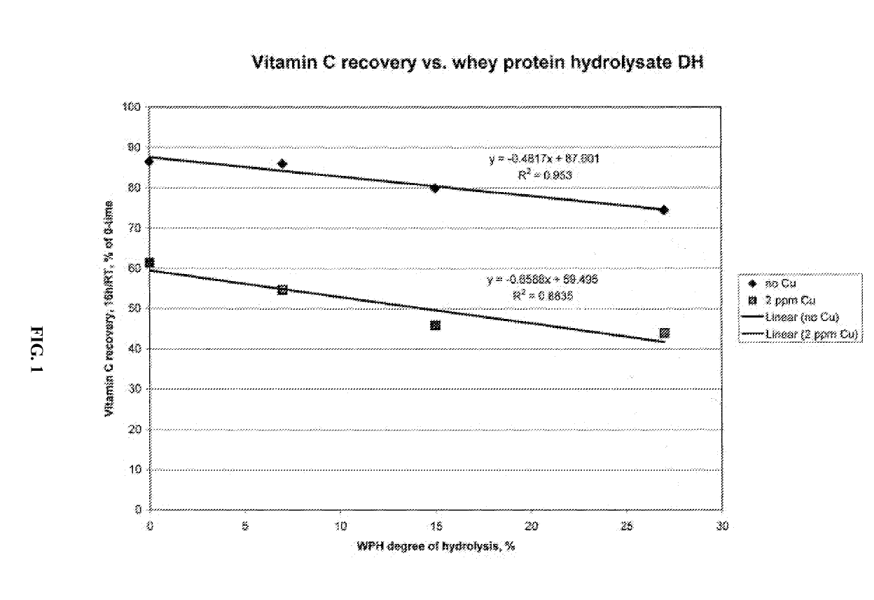

FIG. 1 is a graph illustrating the vitamin C recovery presented in Table 2.

The general inventive concepts encompass liquid nutritional compositions with improved vitamin C stability, as well as methods for improving the stability of vitamin C in liquid nutritional compositions. In certain exemplary embodiments, the liquid nutritional compositions comprise protein in an amount of 1 to 12% by weight of the liquid nutritional composition. In certain exemplary embodiments, the protein comprises 60 to 80% whey protein, and 20 to 40% casein by weight of the protein in the liquid nutritional composition. In certain exemplary embodiments, the liquid nutritional composition comprises 60 to 80% hydrolyzed whey protein by weight of the nutritional composition. In certain exemplary embodiments, the majority of the whey protein in the liquid nutritional composition has a degree of hydrolysis of greater than 20%. In certain exemplary embodiments, the hydrolyzed whey protein is provided in an amount of 1 to 10% by weight of the liquid nutritional composition. In certain exemplary embodiments, 30 to 100% of the total protein in the liquid nutritional composition has a molecular weight below 3000 Daltons.

In certain exemplary embodiments, the liquid nutritional composition comprises vitamin C. In certain exemplary embodiments, vitamin C is provided in an amount of 0.01 to 0.2% by weight of the liquid nutritional composition. The vitamin C may be provided in a single form, or more than one form. In certain exemplary embodiments, the vitamin C is in the form of a salt selected from sodium ascorbate, potassium ascorbate, calcium ascorbate, and magnesium ascorbate.

As mentioned previously, in certain exemplary embodiments, the nutritional composition comprises copper provided in a form that restricts its availability for oxidative processes. Non-limiting forms of copper that restrict its availability for oxidative process include, but are not limited to encapsulation and copper in combination with other compounds that either complex the copper or cause the copper to precipitate from the composition. Disodium phosphate and calcium phosphate (also referred to as tricalcium phosphate or TCP) are suitable compounds that complex the copper, cause the copper to precipitate from the composition, or both. In certain exemplary embodiments, the liquid nutritional composition comprises copper or a source of copper. In certain exemplary embodiments, the liquid nutritional composition comprises copper in an amount of 0.00005 to 0.001% by weight of the liquid nutritional composition. In certain exemplary embodiments, the copper is provided as copper sulfate pentahydrate. In certain exemplary embodiments, the copper is provided in a premix comprising at least one of a dibasic phosphate salt and a tribasic phosphate salt. In certain exemplary embodiments, the premix comprises at least one of disodium phosphate, tricalcium phosphate, sodium phosphate monobasic, potassium phosphate dibasic, potassium phosphate monobasic, dicalcium phosphate, trisodium phosphate, tripotassium phosphate, monocalcium phosphate, and micronized tricalcium phosphate. In certain exemplary embodiments, the liquid nutritional composition comprises sodium tripolyphosphate, disodium phosphate duohydrate, or combinations thereof. In certain exemplary embodiments, the nutritional composition comprises sodium tripolyphosphate and disodium phosphate duohydrate in a ratio of from about 0.1 to about 10. In certain exemplary embodiments, the nutritional composition comprises sodium tripolyphosphate and disodium phosphate duohydrate in a ratio of from about 0.5 to about 5. In certain exemplary embodiments, the nutritional composition comprises sodium tripolyphosphate, disodium phosphate duohydrate in a ratio of from about 0.75 to about 3.

In certain exemplary embodiments, the liquid nutritional composition comprises copper in the form of an encapsulated copper salt. In certain exemplary embodiments, the encapsulated copper salt is encapsulated copper (II) gluconate. In certain exemplary embodiments, the encapsulated copper salt is encapsulated copper (II) sulfate monohydrate. Commercially available sources of encapsulated copper salts are known to those of skill in the art. One such source is available from Dr. Paul Lohmann GmbH.

In certain exemplary embodiments, the liquid nutritional composition is heated. In certain exemplary embodiments, the liquid nutritional composition is heated under a sufficient temperature and for a sufficient period of time to form a sterilized liquid nutritional composition. Liquid nutritional compositions are generally heat sterilized to eliminate microbial life and transmissible agents. When used herein, the term “heat sterilized” refers to the process of heating a liquid nutritional composition to about 260° F. for a time sufficient to kill the microbial life and render the transmissible agents safe.

Liquid nutritional compositions according to certain exemplary embodiments display enhanced vitamin C stability (i.e., decrease vitamin C loss rate, decrease unwanted discoloration, or both). One measure of enhanced vitamin C stability is the percentage of vitamin C present in the liquid nutritional composition after sterilization relative to the amount of vitamin C initially provided in the liquid nutritional composition. In certain exemplary embodiments, the sterilized liquid nutritional composition comprises 40 to 90% vitamin C based on the total vitamin C initially added to the composition.

In certain exemplary embodiments, the nutritional composition further comprises one or more functional ingredients that maintain lean muscle mass, decrease muscle loss, or combinations thereof. For example, in certain exemplary embodiments, the nutritional composition further comprises a functional ingredient selected from the group consisting of: a branched-chain amino acid selected from the group consisting of leucine, isoleucine, valine, metabolites of any of the foregoing branched-chain amino acids including alpha-ketoisocaproic acid and alpha-hydroxyisocaproic acid, alpha-ketoisovaleric acid, alpha-hydroxyisovaleric acid, beta-hydroxyisobutyric acid, 2-oxo-3-methylvaleric acid, 2-hydroxy-3-methylvaleric acid, 3-hydroxy-2-ethylpropionic acid, 3-hydroxy-2-methylbutyric acid, and combinations thereof; creatine; carnitine; carnosine; anserine; taurine; α-ketoglutarate; citrulline; arginine; or a combination thereof.

Different sources and types of proteins, carbohydrate, lipids, vitamins, and minerals, are known and may be used in the exemplary embodiments herein, provided that such nutrients are compatible with the added ingredients in the selected formula, are safe for their intended use, and do not otherwise unduly impair product performance.

In certain exemplary embodiments, the nutritional composition further comprises any of a variety of vitamins, non-limiting examples of which include vitamin A, vitamin K, thiamine, riboflavin, pyridoxine, vitamin B12, niacin, folic acid, pantothenic acid, biotin, inositol, choline, salts and derivatives thereof, and combinations thereof.

In certain exemplary embodiments, the nutritional composition further comprises any of a variety of minerals, non-limiting examples of which include calcium, phosphorus, magnesium, iron, zinc, manganese, copper, iodine, sodium, potassium, molybdenum, chloride, selenium, chromium, chloride, salts and derivatives thereof, and combinations thereof.

In certain exemplary embodiments, the nutritional composition may be an excellent source of (as defined by the Food and Drug Administration) at least one of the following: calcium, iron, riboflavin, vitamin B6, folate, pantothenic acid, phosphorous, iodine, zinc, selenium, manganese, copper, chromium, molybdenum, and combinations thereof.

In certain exemplary embodiments, the nutritional composition may be a good source of (as defined by the Food and Drug Administration) at least one of the following: vitamin A, vitamin C, vitamin E, thiamin, niacin, biotin, and combinations thereof.

In certain exemplary embodiments, the nutritional composition further comprises one or more optional ingredients that may modify the physical, chemical, aesthetic, or processing characteristics of the composition or serve as additional nutritional components when used in the targeted population. Many such optional ingredients are known or otherwise suitable for use in nutritional compositions and may also be used in the nutritional compositions, provided that such optional ingredients are compatible with the essential components described herein, are safe and effective for their intended use, and do not otherwise unduly impair product performance.

Non-limiting examples of such optional ingredients include fiber, preservatives, additional antioxidants, emulsifying agents, buffers, colorants, flavors, probiotics, prebiotics, thickening agents and stabilizers, and so forth.

In certain exemplary embodiments, the nutritional composition comprises a source of fiber. The fiber may be provided by one source, or multiple sources of fiber. The particular amount of fiber present in the nutritional composition may vary depending upon the nutritional needs of the intended user. Fiber is defined as the indigestible portion of food consumed by animals. Dietary fiber is often categorized into soluble and insoluble fiber sources. Non-limiting examples of fibers and sources thereof which may be suitable for use in the exemplary embodiments include: insoluble fibers such as fructooligosaccharides (FOS), galactooligosaccharides (GOS), beta-glucans and lignin; and soluble fibers such as inulin, raffinose, xylose, and polydextrose.

In certain exemplary embodiments, the nutritional composition comprises at least one sweetening agent. In certain exemplary embodiments, the at least one sweetening agent is at least one sugar alcohol such as maltitol, erythritol, sorbitol, xylitol, mannitol, isolmalt, and lactitol, or at least one artificial or high potency sweetener such as acesulfame K, aspartame, sucralose, saccharin, stevia, tagatose, and combinations thereof. The sweetening agents, especially as a combination of a sugar alcohol and an artificial sweetener, may be especially useful in formulating liquid nutritional compositions having a desirable flavor profile. These sweetener combinations can effectively mask undesirable flavors, for example, as sometimes associated with the addition of vegetable proteins to a liquid nutritional composition. In certain exemplary embodiments, the nutritional composition may comprise at least one sugar alcohol with a concentration in a range from at least 0.01%, including from about 0.1% to about 10%, and also including from about 1% to about 6%, by weight of the nutritional composition. In certain exemplary embodiments, the nutritional composition may comprise at least one artificial sweetener with a concentration in a range from 0.01% to 5%, including from 0.05% to 3%, and also including from 0.1% to 1%, by weight of the nutritional composition.

In certain exemplary embodiments, the nutritional composition comprises a stabilizer. Any stabilizer that is known or otherwise suitable for use in a nutritional composition may be suitable for use herein, some non-limiting examples of which include gums such as carageenan, and xanthan gum. In certain exemplary embodiments, the stabilizer may represent from about 0.1% to about 5%, including from about 0.5% to about 3%, and including from about 0.7% to about 1.5%, by weight of the nutritional composition.

In certain exemplary embodiments, the nutritional composition comprises one or more masking agents to reduce or otherwise obscure the effects of any bitter flavors and after taste that may develop in the nutritional compositions over time. Suitable masking agents include natural and artificial sweeteners; sodium sources such as sodium chloride; and combinations thereof. The amount of masking agent added to the nutritional composition may vary depending upon the particular masking agent selected, other ingredients in the formulation, and other formulation or product target variables. Such amounts, however, can range from 0.1% to 3%, including from about 0.15% to about 3%, and also including from about 0.2% to about 2.5%, by weight of the nutritional composition.

The various exemplary embodiments of the nutritional composition disclosed herein, as well as other embodiments contemplated by the general inventive concepts, may be prepared by any process or suitable method (now known or known in the future) for making a selected product form, such as a nutritional liquid, a nutritional solid, or a nutritional powder. Many such techniques are known for any given product form and can easily be applied by one of ordinary skill in the art to the various embodiments presented herein.

In certain exemplary embodiments, the nutritional composition is in the form of a flowable or substantially flowable particulate composition, or at least particulate a composition that may be easily scooped and measured with a spoon or similar device, wherein the composition may be reconstituted by the intended user with a suitable aqueous fluid, such as water, to form a liquid nutritional composition for immediate oral or enteral use by the intended user. In this context, “immediate use” means within about 48 hours, including within about 24 hours, and also including right after reconstitution. The embodiments include spray dried, agglomerated, dry mixed, or other known or otherwise effective particulate forms. The quantity of nutritional powder required to produce a volume suitable for one serving may vary.

In certain exemplary embodiments, the nutritional composition may be packaged and sealed in a single or multi-use container, and then stored under ambient conditions for up to about 36 months, including from about 12 to about 24 months. For a multi-use container, the package may be opened and then covered for repeated use by the user, provided that the covered package is then stored under ambient conditions (e.g., avoiding extreme temperatures) and the entire contents used within about one month or so.

In certain exemplary embodiments, administration of the nutritional composition is helpful to achieve one or more of 1) maintaining lean body mass in obese individuals, 2) providing nutrition to support an active and independent lifestyle in individuals, 3) supporting recovery of muscle strength, 4) helping rebuild muscle and regain strength, and 5) improving strength, including muscle strength, and mobility.

The liquid nutritional compositions may be and typically are shelf stable. The liquid nutritional compositions typically contain up to about 95% by weight of water, including from about 50% to about 95%, also including from about 60% to about 90%, and also including from about 70% to about 85%, of water by weight of the liquid nutritional compositions.

The liquid nutritional compositions may have a pH ranging from about 2.5 to about 8, but are most advantageously in a range of from about 4.5 to about 7.5, including from about 5.5 to about 7.3, and including from about 6.2 to about 7.2.

The liquid nutritional compositions may be manufactured by any known or otherwise suitable method for making liquid nutritional compositions, including emulsions such as milk-based nutritional emulsions.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing the selected oils (e.g., canola oil, corn oil, fish oil) and then adding an emulsifier (e.g., lecithin), fat soluble vitamins, and a portion of the total protein (e.g., milk protein concentrate) with continued heat and agitation. The CHO-MIN slurry is formed by adding with heated agitation to water: minerals (e.g., potassium citrate, dipotassium phosphate, sodium citrate), trace and ultra trace minerals (TM/UTM premix), thickening or suspending agents (e.g. Avicel, gellan, carrageenan), and metal amino acid chelates. The resulting CHO-MIN slurry is held for 10 minutes with continued heat and agitation before adding additional minerals (e.g., potassium chloride, magnesium carbonate, potassium iodide) and/or carbohydrate (e.g., fructooligosaccharide, sucrose, corn syrup). The PIW slurry is then formed by mixing with heat and agitating the remaining protein (e.g., sodium caseinate, soy protein concentrate) into water.

The resulting slurries are then blended together with heated agitation and the pH adjusted to the desired range, typically from 6.6 to 7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range if necessary, flavors are added, and water is added to achieve the desired total solid level. The composition is then aseptically packaged to form an aseptically packaged nutritional liquid, or the composition is added to retort stable containers and then subjected to retort sterilization to form retort sterilized nutritional liquids.

The manufacturing processes for the nutritional liquids may be carried out in ways other than those set forth herein without departing from the spirit and scope of the general inventive concepts. The present embodiments are, therefore, to be considered in all respects illustrative and not restrictive and that all changes and equivalents also come within the description of the exemplary embodiments.

### EXAMPLES

The following examples further describe and demonstrate specific embodiments within the scope of the present invention. The examples are given solely for the purpose of illustration and are not to be construed as limitations of the present invention, as many variations thereof are possible without departing from the spirit and scope of the invention. All exemplified amounts are weight percentages based upon the total weight of the composition, unless otherwise specified.

Example 1 illustrates an exemplary embodiment of a nutritional composition in the form of a nutritional liquid. All ingredient amounts are listed as total amount included in a 1000 pound total batch.

TABLE 3

| | % of | | | |
|---|---|---|---|---|
| Ingredient | Batch | kg | g | lb |
| Water | 77.074% | 349.6053518 | 349605 | 770.7518944 |
| Maltrin 40 | 7.35200% | 33.34845144 | 33348 | 73.52113459 |
| Hydrolysed whey protein | 5.43100% | 24.63485307 | 24635 | 54.31083814 |
| Hydrolyzed casein | 2.59700% | 11.77991409 | 11780 | 25.97040078 |
| Maltrin 100 | 2.45900% | 11.15395023 | 11154 | 24.59037948 |
| Hydrolysed whey protein | 1.13200% | 5.13471804 | 5134.7 | 11.3201747 |
| MCT Oil | 1.05500% | 4.78544835 | 4785.4 | 10.55016281 |
| Marine Oil | 0.8440% | 3.82835868 | 3828.4 | 8.44013025 |
| Sucrose | 0.48600% | 2.20448142 | 2204.5 | 4.860075002 |
| Avicel CL-611 | 0.25000% | 1.1339925 | 1134.0 | 2.500038581 |
| Magnesium Phosphate | 0.17800% | 0.80740266 | 807.40 | 1.78002747 |
| Potassium Citrate | 0.12600% | 0.57153222 | 571.53 | 1.260019445 |
| Calcium Carbonate | 0.12600% | 0.57153222 | 571.53 | 1.260019445 |
| Corn Oil | 0.10600% | 0.48081282 | 480.8 | 1.060016358 |
| Potassium Chloride | 0.10600% | 0.48081282 | 480.81 | 1.060016358 |
| Panodan | 0.10600% | 0.48081282 | 480.81 | 1.060016358 |
| Ascorbic Acid | 0.08000% | 0.3628776 | 362.88 | 0.800012346 |
| Sodium Tripolyphosphate | 0.06500% | 0.29483805 | 294.84 | 0.650010031 |
| Choline Chloride | 0.06100% | 0.27669417 | 276.69 | 0.610009414 |
| m TriCalcium Phosphate | 0.06000% | 0.2721582 | 272.16 | 0.600009259 |
| 45% Potassium Hydroxide | 0.05600% | 0.25401432 | 254.01 | 0.560008642 |
| Disodium Phosphate | 0.05000% | 0.2267985 | 226.80 | 0.500007716 |
| Iota Carrageenan | 0.04500% | 0.20411865 | 204.12 | 0.450006945 |
| DEK Premix | 0.04410% | 0.200036277 | 200.04 | 0.441006806 |
| Magnesium Chloride | 0.02600% | 0.11793522 | 117.94 | 0.260004012 |
| UTM/TM Premix | 0.02600% | 0.11793522 | 117.94 | 0.260004012 |
| WSV Premix | 0.02425% | 0.109997273 | 110.00 | 0.242503742 |
| Taurine | 0.01323% | 0.060010883 | 60.011 | 0.132302042 |
| Carnitine | 0.01323% | 0.060010883 | 60.011 | 0.132302042 |
| Anti-foam | 0.00600% | 0.02721582 | 27.216 | 0.060000926 |
| Vitamin A Palmitate | 0.00100% | 0.00453597 | 4.5360 | 0.010000154 |
| Potassium Iodide | 0.00002% | 8.61834E−05 | 0.086 | 0.000190003 |
| Total | | | | 1000.00 |

All percentages, parts, and ratios as used herein are by weight of the total composition, unless otherwise specified. All such weights as they pertain to listed ingredients are based on the active level and, therefore, do not include solvents or by-products that may be included in commercially available materials, unless otherwise specified.

All references to singular characteristics or limitations of the embodiments described herein shall include the corresponding plural characteristic or limitations, and vice versa, unless otherwise specified or clearly implied to the contrary by the context in which the reference is made.

All combinations of method or process steps as used herein may be performed in any order, unless otherwise specified or clearly implied to the contrary by the context in which the referenced combination is made.

The embodiments herein may also be substantially free of any optional or selected essential ingredient or feature described herein, provided that the remaining formula still contains all of the required ingredients or features as described herein. In this context, and unless otherwise specified, the term “substantially free” means that the selected composition contains less than a functional amount of the optional or selected ingredient, typically less than 0.1% by weight, and also including zero percent by weight of such optional or selected ingredient.

The embodiments described herein may comprise, consist of, or consist essentially of the essential elements and limitations of the invention described herein, as well as any additional or optional ingredients, components, or limitations described herein or otherwise useful in nutritional formula applications.

1. A liquid nutritional composition comprising protein, carbohydrate, and fat; wherein the fat is present in an amount of 15 to 25% of the total calories of the liquid nutritional composition; and wherein the fat comprises a source of at least one omega-6 fatty acid and a source of at least one omega-3 fatty acid, with a ratio of omega-6 to omega-3 fatty acids of 1:5 to 1:1.

2. The liquid nutritional composition of claim 1, the fat comprising a source of at least one omega-3 fatty acid selected from eicosapentaenoic acid and docosahexaenoic acid.

3. The liquid nutritional composition of claim 1, wherein the ratio of omega-6 to omega-3 fatty acids is 1:4.5 to 1:3.

4. The liquid nutritional composition of claim 1, wherein the ratio of omega-6 to omega-3 fatty acids is 1:4.

5. The liquid nutritional composition of claim 1, wherein the fat is present in an amount of not more than 20% of the total calories of the liquid nutritional composition.

6. The liquid nutritional composition of claim 1, wherein the fat is present in an amount of 20% of the total calories of the liquid nutritional composition.

7. The liquid nutritional composition of claim 1, wherein the protein is present in an amount of 30 to 40% of the total calories of the liquid nutritional composition.

8. The liquid nutritional composition of claim 1, wherein the protein is present in an amount of not more than 35% of the total calories of the liquid nutritional composition.

9. The liquid nutritional composition of claim 1, wherein the protein comprises at least one dairy protein.

10. The liquid nutritional composition of claim 1, wherein the protein comprises at least one of a hydrolyzed whey protein and a hydrolyzed casein protein.

11. The liquid nutritional composition of claim 1, wherein the protein comprises hydrolyzed whey protein, the hydrolyzed whey protein having a degree of hydrolysis of 20% to 70%.

12. The liquid nutritional composition of claim 1, wherein the protein comprises hydrolyzed casein protein, the hydrolyzed casein protein having a degree of hydrolysis of 0.1% to 10%.

13. (canceled)

14. The liquid nutritional composition of claim 1, wherein the carbohydrate is present in an amount of 35 to 55% of the total calories of the liquid nutritional composition.

15. The liquid nutritional composition of claim 1, wherein the carbohydrate is present in an amount of at least 40% of the total calories of the liquid nutritional composition.

16. (canceled)

17. The liquid nutritional composition of claim 1, wherein the liquid nutritional composition comprises 0 g/L to 1 g/L of indigestible carbohydrate.

18. (canceled)

19. The liquid nutritional composition of claim 1, wherein the liquid nutritional composition comprises at least one of eicosapentaenoic acid, docosahexaenoic acid, and combinations thereof, in an amount of at least 2 g/L of the liquid nutritional composition.

20.-22. (canceled)

23. The liquid nutritional composition of claim 1, wherein the fat comprises a ratio of medium chain triglycerides to long chain triglycerides of 1:1.

24. The liquid nutritional composition of claim 1, wherein the liquid nutritional composition has a caloric density of 1 kcal per mL.

25. (canceled)

26. A method of treating an individual suffering from obesity, the method comprising: administering, a liquid nutritional composition comprising protein, carbohydrate, and fat to the individual; wherein the fat is present in an amount of 15 to 25% of the total calories of the liquid nutritional composition; and wherein the fat comprises a source of at least one omega-6 fatty acid and a source of at least one omega-3 fatty acid; with a ratio of omega-6 to omega-3 fatty acids of 1:5 to 1:1.

27.-44. (canceled)

45. A composition useful for treating a subject suffering from obesity, the composition comprising: protein present in an amount of 30 to 40% of the total calories of the liquid nutritional composition, the protein comprising a source of at least one dairy protein selected from hydrolyzed whey protein and hydrolyzed casein protein; carbohydrate present in an amount of 35 to 55% of the total calories of the liquid nutritional composition, the carbohydrate comprising less than 1 g/L of indigestible carbohydrate; and fat present in an amount of 15 to 25% of the total calories of the liquid nutritional composition; wherein the fat comprises a source of at least one omega-6 fatty acid and a source of at least one omega-3 fatty acid, with a ratio of omega-6 to omega-3 fatty acids of 1:5 to 1:1.

46. (canceled)

47. A liquid nutritional composition comprising: protein in an amount of 1 to 12% by weight of the liquid nutritional composition, vitamin C in an amount of 0.01 to 0.2% by weight of the liquid nutritional composition, and copper in an amount of 0.00005 to 0.001% by weight of the liquid nutritional composition, wherein the protein comprises 60 to 80% whey protein, and 20 to 40% casein by weight of the protein in the liquid nutritional composition, with a majority of the whey protein having a degree of hydrolysis greater than 20%.

48.-74. (canceled)

75. A method for improving vitamin C stability in a liquid nutritional composition, the method comprising: providing hydrolyzed whey protein in an amount of 1 to 10% by weight of the liquid nutritional composition, a majority of the hydrolyzed whey protein having a degree of hydrolysis greater than 20%; providing a combination of vitamin C in an amount of 0.01 to 0.2% by weight of the liquid nutritional composition and copper in an amount of 0.00005 to 0.001% by weight of the liquid nutritional composition; and mixing the hydrolyzed whey protein, vitamin C, and copper to form a liquid nutritional composition, wherein the stability of the vitamin C is enhanced.

76.-86. (canceled)
